(19) United States
(12) Patent Application Publication
(10) Pub. No.: US 2007/0167684 A1
(43) Pub. Date: Jul. 19, 2007

(54) Endoscope device, endoscope system, and method for inserting endoscope device into body cavity

(75) Inventor: Ryuichi Toyama, Tokyo (JP)

Correspondence Address: Thomas Spinelli;Scully, Scott, Murphy & Presser, Suite 300, 400 Garden City Plaza, Garden City, NY, 11530, US

(73) Assignee: Olympus Medical Systems Corp., Tokyo (JP)

(21) Appl. No.: 11/648,995

(22) Filed: Jan. 3, 2007

(30) Foreign Application Priority Data
Jan. 13, 2006 (JP) 2006-006796

(51) Int. Cl. A61B 1/00

(52) U.S. Cl. 600/128; 600/139; 600/114

(57) ABSTRACT

An endoscope device includes an insertion portion cover as an insertion portion main body insertable into an examinee's body and having flexibility, and a helical shaped portion as a propulsion force generating portion which is rotatably placed around an outer circumference of the insertion portion cover, centered around the axis thereof, and formed in a clockwise winding toward a distal end side of the insertion portion cover. The endoscope device can thus achieve good operationality of the insertion portion, low cost, and improved insertability into a deep part in a body cavity without inflicting a pain on a patient.

### CROSS REFERENCE TO RELATED APPLICATIONS

This application claims benefit of Japanese Application No. 2006-6796 filed on Jan. 13, 2006, the contents of which are incorporated by this reference.

### BACKGROUND OF THE INVENTION

1. Field of the Invention

The present invention relates to an endoscope device including an insertion portion main body having flexibility which is insertable into an examinee's body, an endoscope system, and a method for inserting the endoscope device into a body cavity.

2. Description of the Related Art

Conventionally, medical endoscope devices have been widely used. With the medical endoscope device, an elongate insertion portion is inserted into a body cavity to observe a diseased part or the like in the body cavity, and a treatment tool is inserted through a forceps channel as required to allow a curing treatment to be performed. The endoscope device has a bendable bending portion at a distal end side of the insertion portion. In the endoscope device, the bendable bending portion is bent and operated in up/down or left/right directions by operating a bending operation knob.

When the endoscope device is inserted into a convoluted intracavital canal, e.g., a lumen forming a loop of 360 degrees such as the large intestine, the bending operation knob is operated to bend and operate the bending portion, while the insertion portion is twisted and operated to be inserted toward an observation target position. However, the operation of the endoscope requires mastery to be able to insert the insertion portion smoothly in a short period of time into a deep part in the convoluted large intestine. It was concerned that an inexperienced surgeon, lost in the insertion direction, has trouble in inserting the insertion portion into the deep part in the large intestine, or greatly change the way the intestine runs.

For this reason, various proposals have conventionally been made for improving the insertability of the insertion portion. For example, Japanese unexamined patent publication No. 10-113396 discloses a propulsion device for medical apparatus capable of easily and low-invasively guiding a medical apparatus into a deep part of an intracavital canal. The propulsion device has a rotation member provided in the axial direction thereof with a diagonal rib serving as a propulsion force generating portion. Thus, in the propulsion device described in the publication, with rotational motion of the rotation member, rotational force of the rotation member is converted to a propulsion force by the rib, and the medical apparatus connected to the propulsion device is moved toward the deep part of the intracavital canal by the propulsion force. In this manner, the propulsion device described in Japanese unexamined patent publication No. 10-113396 can insert a medical apparatus into a body cavity low-invasively without inflicting a patient with a physical burden.

### SUMMARY OF THE INVENTION

An endoscope device according to one aspect of the present invention includes: an insertion portion main body having flexibility which is insertable into an examinee's body; a rotation propulsion portion rotatably placed around an outer circumference near a distal end of the insertion portion main body and centered around an axis of the insertion portion main body, the rotation propulsion portion having on an outer circumference thereof a helical shaped portion serving as a propulsion force generating portion; and a rotation transmission shaft having flexibility for transmitting rotation force to the rotation propulsion portion from a proximal end side of the insertion portion main body, wherein the helical shaped portion has a helix formed in a direction to exhibit a propulsion force to the direction of the distal end of the insertion portion when the rotation transmission shaft is rotated clockwise toward the distal end of the insertion portion main body.

An endoscope system according to another aspect of the present invention includes: an endoscope device including: an insertion portion main body having flexibility which is insertable into an examinee's body; a rotation propulsion portion rotatably placed around an outer circumference near a distal end of the insertion portion main body and centered around an axis of the insertion portion main body, the rotation propulsion portion having on an outer circumference thereof a helical shaped portion serving as a propulsion force generating portion; and a rotation transmission shaft having flexibility for transmitting rotation force to the rotation propulsion portion from a proximal end side of the insertion portion main body, the helical shaped portion being helically formed in a direction to exhibit propulsion force toward the distal end of the insertion portion when the rotation transmission shaft is rotated clockwise toward the distal end of the insertion portion main body; and a rotation device for rotating the helical shaped portion of the endoscope device about a longitudinal axis of the helical shaped portion.

A method for inserting an endoscope device into a body cavity according to yet another aspect of the present invention includes: inserting an endoscope device into an opening of an intracavital canal, the endoscope device including: an insertion portion main body having flexibility which is insertable into an examinee's body; a rotation propulsion portion placed around an outer circumference near a distal end of the insertion portion main body and centered around an axis of the insertion portion main body, the rotation propulsion portion having on an outer circumference thereof a helical shaped portion serving as a propulsion force generating portion; and a rotation transmission shaft having flexibility for transmitting rotation force to the rotation propulsion portion from a proximal end side of the insertion portion main body, the helical shaped portion having a helix formed in a direction to exhibit a propulsion force to the direction of the distal end of the insertion portion when the rotation transmission shaft is rotated clockwise toward the distal end of the insertion portion main body; rotating the helical shaped portion of the endoscope device inserted into the opening of the intracavital canal, clockwise about a longitudinal axis of the insertion portion main body; propelling the insertion portion main body toward a deep part of the intracavital canal, by obtaining a propulsion force between the helical shaped portion and an inner wall of the intracavital canal by the helical shaped portion which is rotating clockwise about the longitudinal axis of the insertion portion main body; and generally linearizing the body cavity, by propelling the endoscope device toward the deep part of the intracavital canal while drawing the body cavity to right side of the examinee's body by means of friction action of the helical shaped portion rotating clockwise about the longitudinal direction of the insertion portion main body so as to shorten the body cavity.

The endoscope device and the endoscope system according to these inventions have good operationality of the insertion portion, are low-cost, and have an effect of being capable of improving the insertability to the deep part of the intracavital canal without inflicting a patient with pain.

The above and other objects, features, and advantages of the inventions will become more clearly understood from the following description referring to the accompanying drawings.

### BRIEF DESCRIPTION OF THE DRAWINGS

FIG. 1 is a general configuration view showing an endoscope system of one embodiment of the present invention.

FIG. 2 is an external view showing a vicinity of a distal end portion of an introduction tube of FIG. 1.

FIG. 3 is an illustrative view showing the introduction tube and an endoscope of FIG. 1.

FIG. 4 is a cross-sectional view taken along the line IV-IV of FIG. 3.

FIG. 5 is an illustrative view showing a configuration of a rotation mechanism portion.

FIG. 6 is an illustrative view of a main portion showing a vicinity of the distal end portion of the introduction tube of FIG. 2.

FIG. 7 is an illustrative view showing a state where an introduction tube in which an insertion portion is introduced and placed is introduced from the anus.

FIG. 8 is an illustrative view showing a state where the distal end portion of the introduction tube contacts the Sigmoid colon portion from the state shown in FIG. 7, thereby interrupting the rotation of a helical tube.

FIG. 9 is an illustrative view showing a situation where the Sigmoid colon portion is applied with a rotation force from the helical tube in the state shown in FIG. 8.

FIG. 10 is an illustrative view showing a situation where the distal end portion of the introduction tube is moving forward in the Sigmoid colon portion from the state shown in FIG. 9.

FIG. 11 is an illustrative view showing a situation where the distal end portion of the introduction tube is moving forward via the Sigmoid colon portion from the state shown in FIG. 10.

FIG. 12 is an illustrative view showing a situation where the distal end portion of the introduction tube is moving forward to the descending colon portion from the state shown in FIG. 11.

FIG. 13 is an illustrative view showing a situation where the distal end portion of the introduction tube has reached the hepatic flexure from the state shown in FIG. 12, and the transverse colon portion, the splenic flexure, and the hepatic flexure are generally applied with rotation force from the helical tube.

FIG. 14 is an illustrative view of a situation where the transverse colon portion, the splenic flexure, and the hepatic flexure are generally linearized from the state shown in FIG. 13.

FIG. 15 is an illustrative view showing a modified example of the introduction tube.

FIG. 16 is a cross-sectional view taken along the line XVI-XVI of FIG. 15.

### DETAILED DESCRIPTION OF PREFERRED EMBODIMENTS

An embodiment of the present invention will be described below with reference to the drawings.

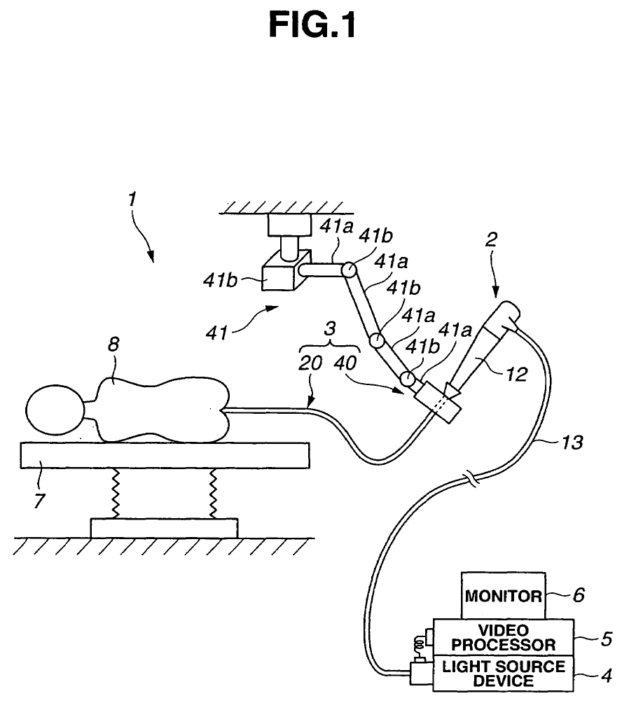

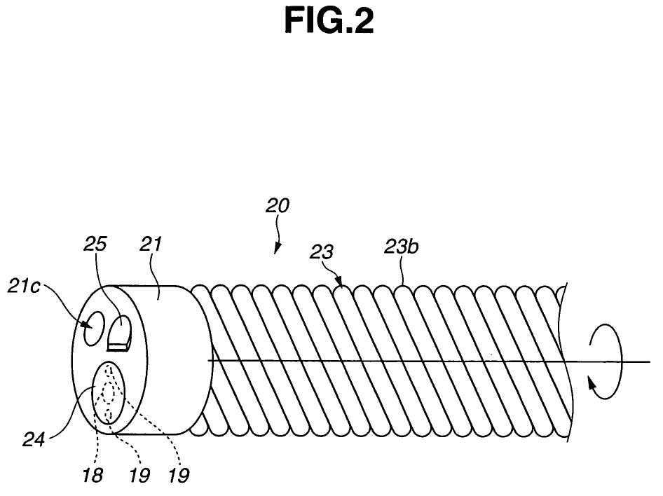

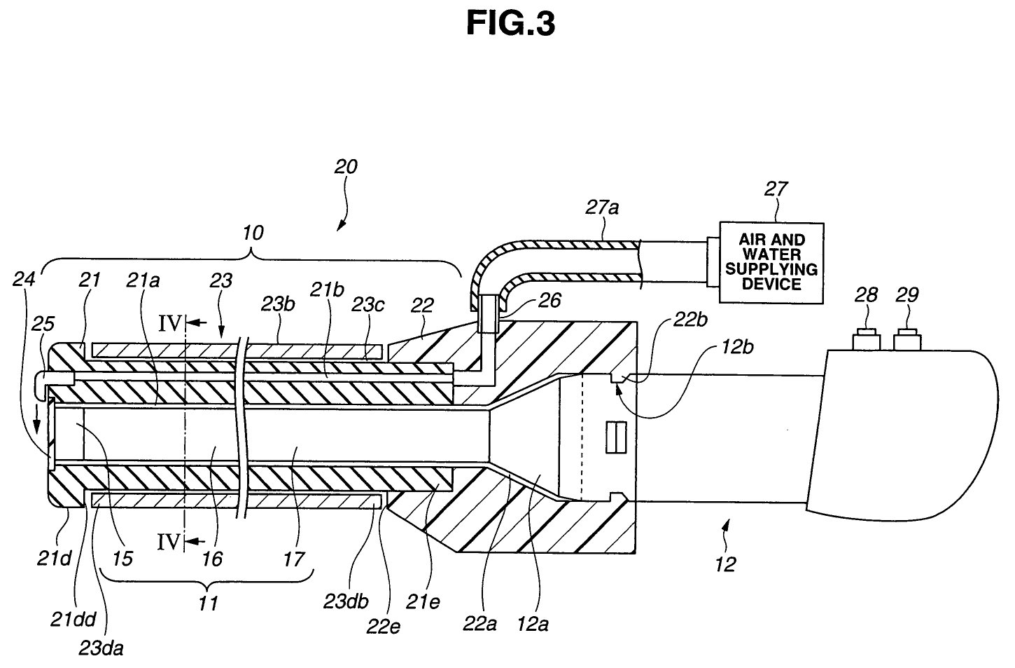

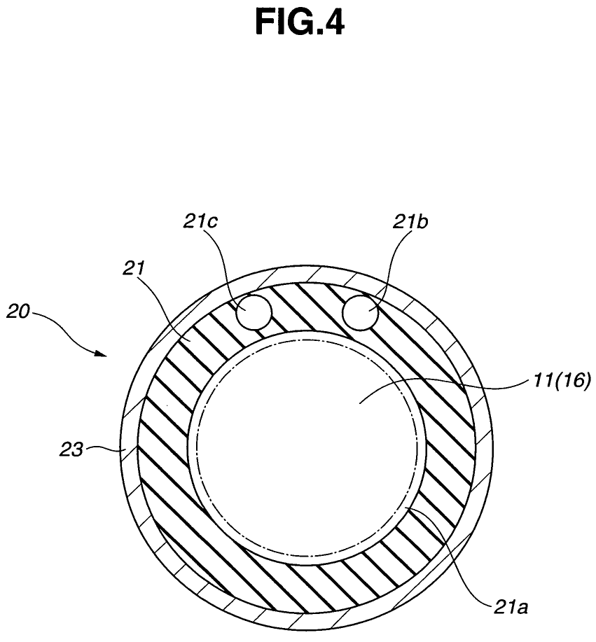

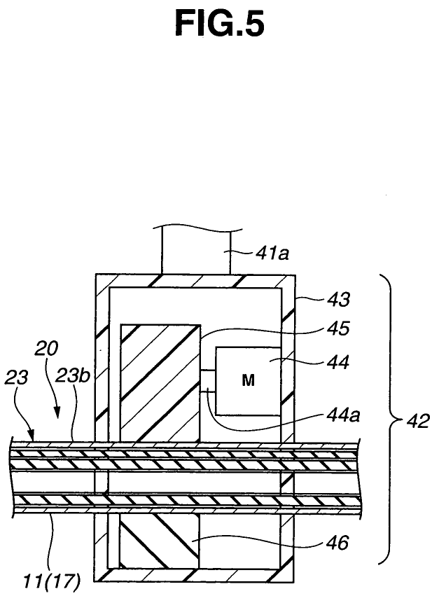

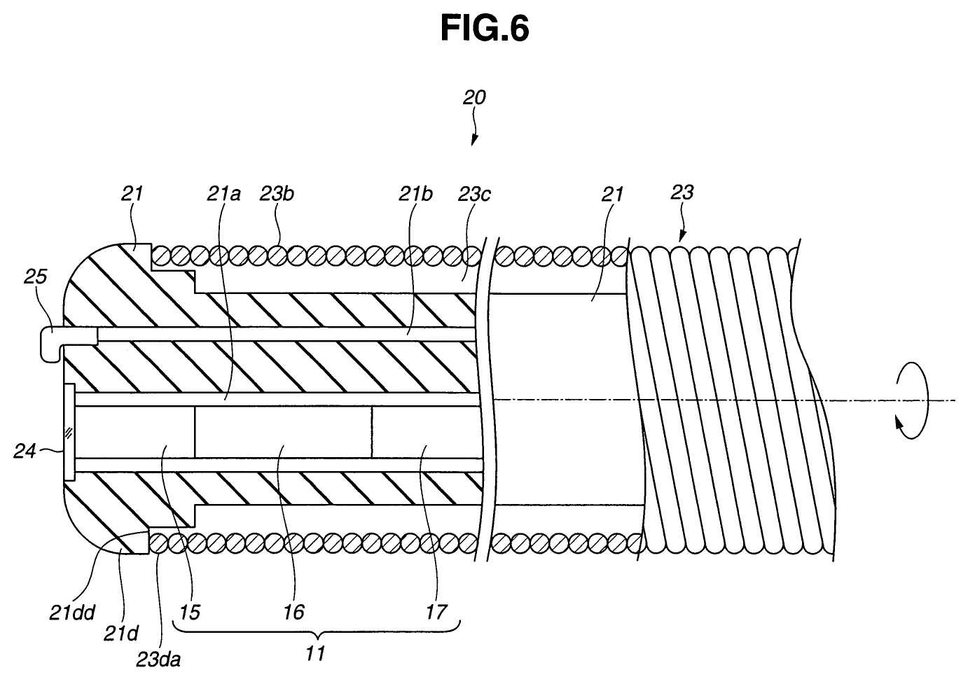

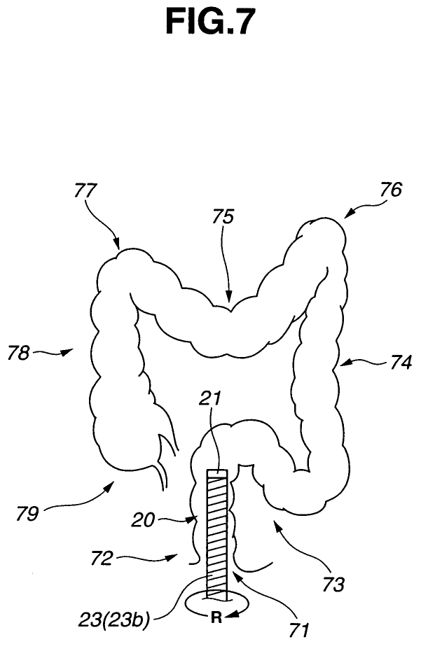

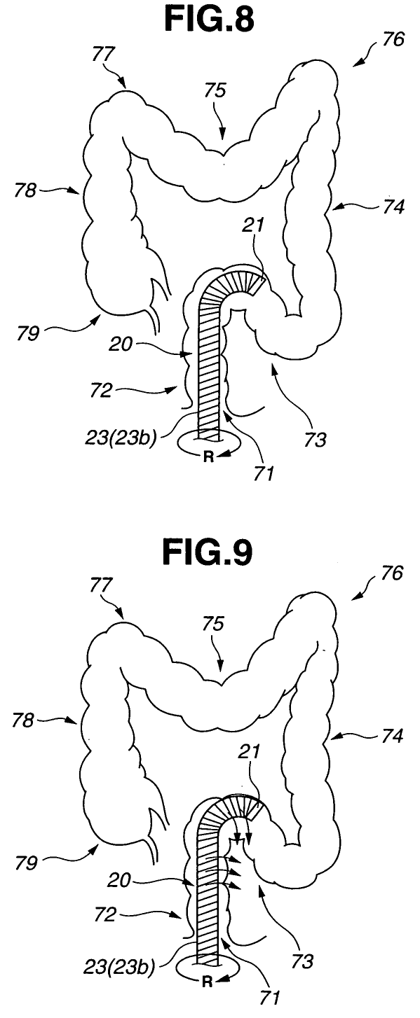

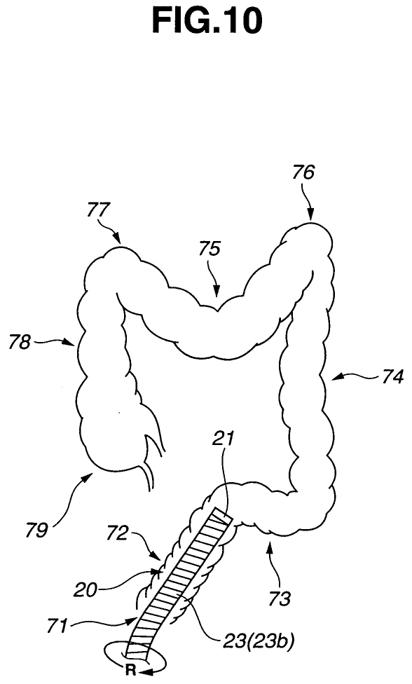

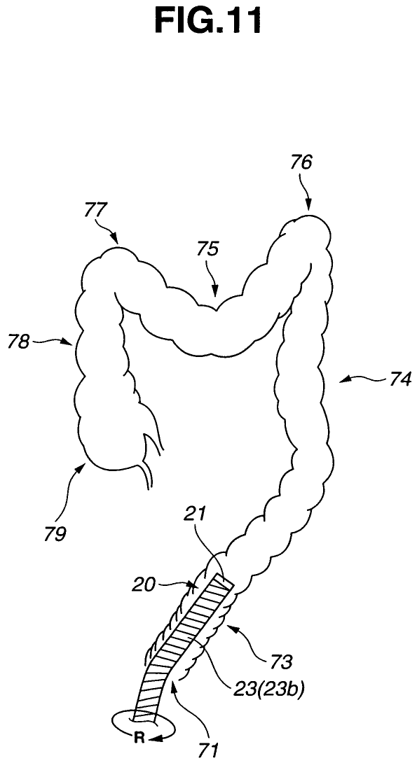

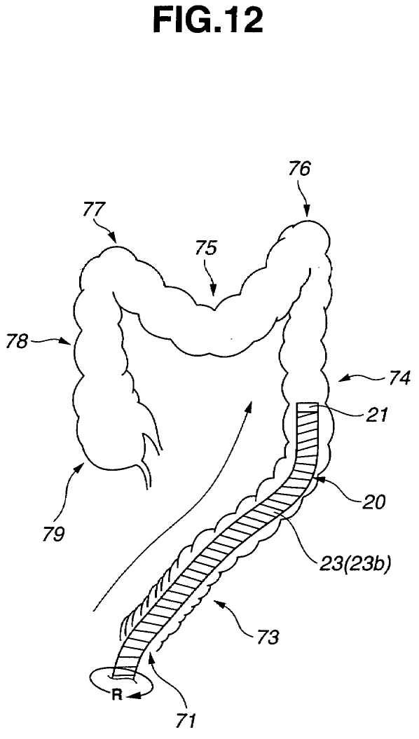

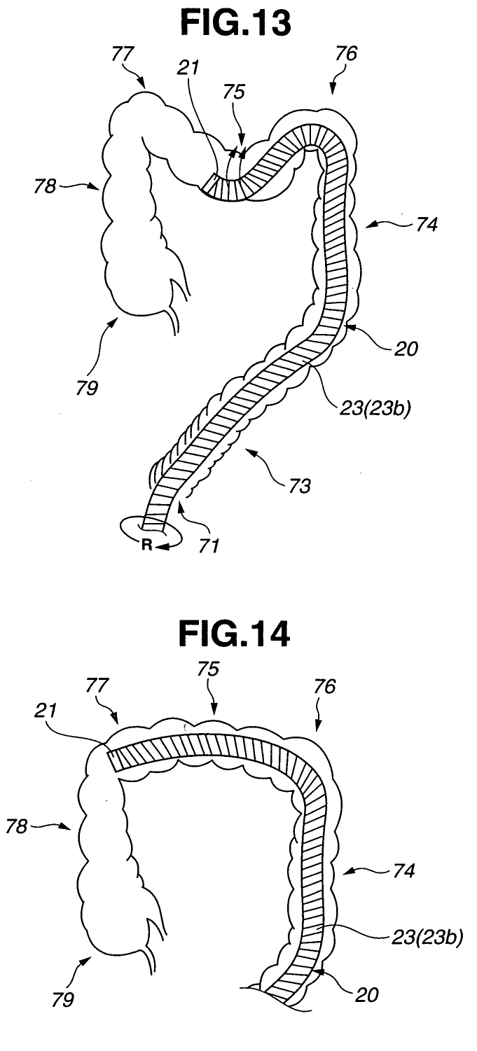

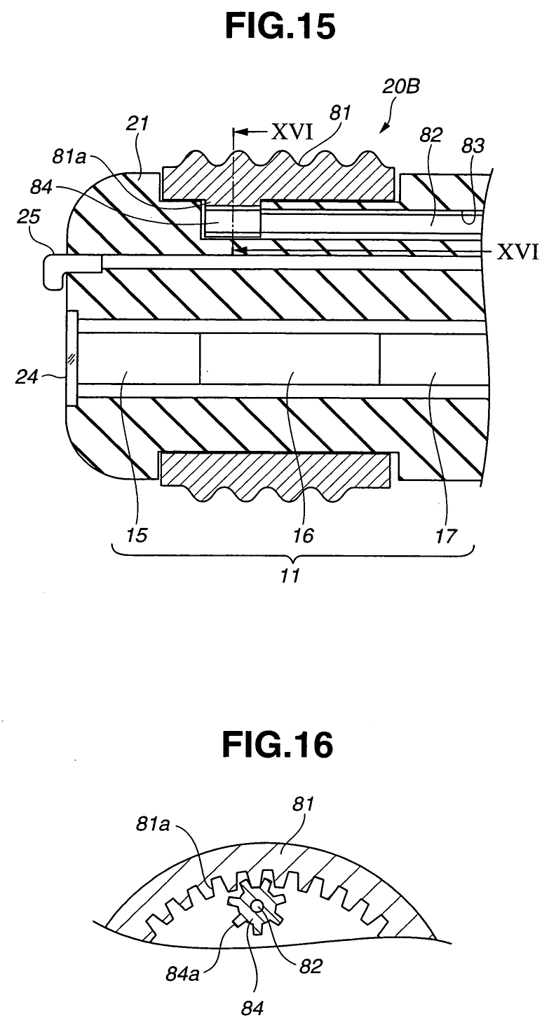

FIGS. 1 to 16 relate to the embodiment of the present invention, wherein: FIG. 1 is a general configuration view showing an endoscope system; FIG. 2 is an external view showing a vicinity of a distal end portion of an introduction tube of FIG. 1; FIG. 3 is an illustrative view showing the introduction tube and an endoscope of FIG. 1; FIG. 4 is a cross-sectional view taken along the line IV-IV of FIG. 3; FIG. 5 is an illustrative view showing a configuration of a rotation mechanism portion; FIG. 6 is an illustrative view of a main portion showing a vicinity of the distal end portion of the introduction tube of FIG. 2; FIG. 7 is an illustrative view showing a state where an introduction tube in which an insertion portion is introduced and placed is introduced from the anus; FIG. 8 is an illustrative view showing a state where the distal end portion of the introduction tube contacts the Sigmoid colon portion from the state shown in FIG. 7, thereby interrupting the rotation of a helical tube; FIG. 9 is an illustrative view showing a situation where the Sigmoid colon portion is applied with a rotation force from the helical tube in the state shown in FIG. 8; FIG. 10 is an illustrative view showing a situation where the distal end portion of the introduction tube is moving forward in the Sigmoid colon portion from the state shown in FIG. 9; FIG. 11 is an illustrative view showing a situation where the distal end portion of the introduction tube is moving forward via the Sigmoid colon portion from the state shown in FIG. 10; FIG. 12 is an illustrative view showing a situation where the distal end portion of the introduction tube is moving forward to the descending colon portion from the state shown in FIG. 11; FIG. 13 is an illustrative view showing a situation where the distal end portion of the introduction tube has reached hepatic flexure from the state shown in FIG. 12, and the transverse colon portion, the splenic flexure, and the hepatic flexure are generally applied with rotation force from the helical tube; FIG. 14 is an illustrative view of a situation where the transverse colon portion, the splenic flexure, and the hepatic flexure are generally linearized from the state shown in FIG. 13; FIG. 15 is an illustrative view showing a modified example of the introduction tube; and FIG. 16 is a cross-sectional view taken along the line XVI-XVI of FIG. 15.

As shown in FIGS. 1 to 4, an endoscope system 1 includes an endoscope 2 and an endoscope insertion-assisting apparatus 3. The endoscope 2 is connected to a light source device 4 for supplying illumination light, a video processor 5, a monitor 6, and the like, which are external devices. The endoscope 2 is supplied with illumination light from the light source device 4, and illuminates a subject with the illumination light. The endoscope 2 captures an image of the illuminated subject from an object optical system not shown, and then outputs an image-capturing signal obtained by photoelectrically converting the captured image with an image-capturing element, to the video processor 5. The video processor 5 signal-processes the image-capturing signal from the image-capturing element to generate a video signal, and outputs and displays the signal to the monitor 6. Note that the endoscope 2 may not be provided with the light source device 4, and instead a distal end portion 15 may be provided with an illumination portion such as LEDs.

The endoscope 2 includes an endoscope insertion portion 11 which is elongate and has flexibility, an operation portion 12 provided on a proximal end side of the endoscope insertion portion 11, and a universal code 13 extending from a side portion of the operation portion 12. The endoscope insertion portion 11 is constructed by connecting in series the rigid distal end portion 15, a bendable bending portion 16, and a flexible tube portion 17 which is long and has flexibility, in this order from the distal end side.

In the operation portion 12 is provided a bending operation knob not shown for bending and operating the bending portion 16. In the endoscope 2, the bending portion 16 is bent and operated in a freely bendable manner by operating the bending operation knob. Note that an introduction tube 20 to be described later in which the endoscope 2 is to be inserted and placed is constructed to bend following the bending motion of the bending portion 16 of the endoscope 2.

The endoscope insertion-assisting apparatus 3 includes the introduction tube 20 in which the endoscope insertion portion 11 is inserted and placed so as to guide the endoscope insertion portion 11 toward a deep part in the body cavity, and a rotation device 40 for rotating a helical tube 23 to be described later of the introduction tube 20.

The rotation device 40 includes, e.g., an arm portion 41 having one end portion attached to, e.g., the ceiling of an inspection room, and a rotation mechanism portion 42 attached to the other end portion of the arm portion 41. The arm portion 41 includes a plurality of arm members 41a differing, e.g., in length, and joint portions 41b for rotatably connecting the adjacent arm members 41a. This permits the rotation device 40 to move the position of the rotation mechanism portion 42 to any position with a small amount of force. Detailed configuration of the rotation mechanism portion 42 will be described later.

As shown in FIGS. 2 to 4, the introduction tube 20 includes: an insertion portion cover 10 serving as an insertion portion main body which is formed from an observation window member 24 and an elastic cover tube 21; a proximal-side component member 22 provided continuously to the insertion portion cover 10; and a helical tube 23 which is placed around an outer circumferential side of the insertion portion cover 10 and forms a helical shaped portion 23b serving as a propulsion force generating portion for generating a propulsion force.

That is, the insertion portion cover 10 serving as the insertion portion main body is equipped with the helical tube 23 which is placed around the outer circumferential surface side and forms the helical shaped portion 23b serving as the propulsion force generating portion that rotates about the longitudinal axis thereof. The helical tube 23 constructs a rotation propulsion portion and a rotation transmission shaft.

The elastic cover tube 21 is formed in an elongate tubular shape by a member having a small frictional resistance, e.g., fluorocarbon resin such as PTFE (polytetrafluoroethylene resin) and the like. The elastic cover tube 21 is formed with a through-hole 21 a penetrating therethrough in the axial direction in which the endoscope insertion portion 11 is to be inserted and placed.

Also, in the elastic cover tube 21, a channel 21b serving as an air and water supplying duct is formed in the axial direction. Further, in the elastic cover tube 21, a channel 21c serving as a treatment tool insertion duct or a suction duct is formed in the axial direction, as shown in FIG. 4.

At a front surface on the distal end side of the elastic cover tube 21, the observation window member 24 is placed to an opening on a distal end side of the through-hole 21a, integrally with the elastic cover tube 21 by adhesion or the like. A proximal end side of the through-hole 21a communicates with a penetration hole 22a to be described later which is formed in the proximal-side component member 22.

The observation window member 24 is formed by a transparent resin member, e.g., polycarbonate and the like, having an optical characteristic. The observation window member 24 has an inner surface to be contacted with a front surface of the distal end portion 15 constructing a part of the endoscope insertion portion 11 when the endoscope insertion portion 11 is inserted and placed in the through-hole 21a. The observation window member 24 serves to water-tightly seal the front opening of the elastic cover tube 21, and as an observation window of the endoscope 2.

The channel 21b has one end side communicating with an air and water supplying nozzle 25 placed near the distal end portion of the elastic cover tube 21. The air and water supplying nozzle 25 has an opening which is opposed to the observation window member 24. On the other end side of the channel 21b is provided a clasp portion 26 projecting from an outer circumference of the proximal-side component member 22.

To the clasp portion 26, one end of an air and water supplying tube 27a is connected. The other end of the air and water supplying tube 27a is connected with an air and water supplying device 27. The air and water supplying device 27 can be driven and controlled through press-operating an air and water supplying pressing button-switch 28.

The air and water supplying device 27 can be driven by press-operating the air and water supplying pressing button-switch 28, to supply a fluid such as air and liquid to the channel 21b to spout out the fluid from the opening of the air and water supplying nozzle 25 to the surface of the observation window member 24, as shown with an arrow.

With this, when the surface of the observation window member 24 is adhered with, e.g., a filth and the like, the introduction tube 20 can wash away the adhering filth by spouting out water from the opening of the air and water supplying nozzle 25. Moreover, the introduction tube 20 can remove beads of moisture adhering on the surface of the observation window member 24 by supplying air from the opening of the air and water supplying nozzle 25.

The channel 21c communicates with a channel opening portion formed at a predetermined position of the proximal-side component member 22. When the channel 21c is used as a treatment tool insertion channel, a treatment tool, e.g., a biopsy needle, biopsy forceps, and so on, is inserted to the channel opening portion.

The treatment tool is inserted through the channel 21c and projects from a distal end opening of the elastic cover tube 21, thus allowing a prescribed treatment to be performed. When the channel 21c is used as a suction channel, one end of a channel connection member is provided and placed to the channel opening portion, and the other end of the channel connection member is connected to a suction duct (not shown) extended from, e.g., a suction device (not shown).

The suction device can be driven and controlled by press-operating a suction pressing button-switch 29. With this, the introduction tube 20 can suck body fluid and the like in the body cavity from the distal end opening of the elastic cover tube 21 by the sucking operation of the suction device.

Accordingly, in the endoscope 2, a distal end surface of the endoscope insertion portion 11 is only provided with an observation window 18 constructing an observation optical system and an illumination window 19 constructing an illumination optical system, in order to reduce the diameter of the endoscope insertion portion 11.

The helical tube 23 is formed by winding a metal wire with a predetermined diameter dimension in a helical shape to have a predetermined flexibility. The metal wire is made of, e.g., stainless. Thus, on the outer surface of the helical tube 23, the helical shaped portion 23b is formed by the surface of the metal wire. The helical tube 23 covers the circumferential surface of the elastic cover tube 21 with a gap 23c formed between an inner circumferential surface of the helical shaped portion 23b and the outer circumferential surface of the elastic cover tube 21, and is placed rotatably in a circumferential direction (about the axis) with respect to the outer circumferential surface of the elastic cover tube 21. Note that the helical tube 23 rotates in the circumferential direction (about the axis) by the rotation mechanism portion 42 of the rotation device 40, as will be described later.

The helical tube 23 is not limited to one-stria construction, but may be formed by a winding in multiple striae, e.g., two or four striae, and the like. Also, the helical tube 23 can be adjusted in propulsion force, progression speed, and so forth, by varying the density of the metal wire and making various helical angle settings when helically winding the metal wire.

On a distal end portion of the outer circumferential surface of the elastic cover tube 21, a convex portion 21 d is provided for preventing the helical tube 23 from dropping off. The helical tube 23 is restricted in its forward movement in that a front end portion 23da contacts and is stopped by a rear surface portion 21dd of the convex portion 21d.

The helical tube 23 is also restricted in its backward movement in that a rear end portion 23db contacts and is stopped by a front surface portion 22e of the proximal-side component member 22. Accordingly, the helical tube 23 always maintain the state of covering the outer circumferential side of the elastic cover tube 21, in that the front end portion 23da is stopped by the rear surface portion 21dd of the convex portion 21d on the front end side, and the rear end portion 23db by the front surface portion 21e of the proximal-side component member 22 on the rear end side.

On the other hand, the proximal-side component member 22 is a tubular member larger in diameter than the elastic cover tube 21, and is formed by a resin member with a good slidability, e.g., Polyacetal and the like. Inside the proximal-side component member 22, the penetration hole 22a is bored and provided in which a part of the distal end side of the operation portion 12 of the endoscope 2 (part of a breaking prevention portion 12a) is to be inserted and placed.

On an inner circumferential surface on a rear end side of the penetration hole 22a, a plurality of inwardly projecting stopping convex portions 22b are projectingly provided. The plurality of stopping convex portions 22b are configured to fit in a circumferential groove 12b formed to the breaking prevention portion 12a of the operation portion 12 of the endoscope 2.

With this, the introduction tube 20 fixes and holds the endoscope 2 in that the plurality of stopping convex portions 22b fits in the circumferential groove 12b when the endoscope insertion portion 11 is inserted inside the elastic cover tube 21 and a part of the distal end side of the operation portion 12 is placed inside the proximal-side component member 22.

In addition, in the front surface portion 22e of the proximal-side component member 22, a part of a proximal end portion 21e of the elastic cover tube 21 fits. Thus, the elastic cover tube 21 is formed to integrate with the proximal-side component member 22.

As shown in FIG. 5, the rotation mechanism portion 42 has a rotation portion main body 43 which is a housing, a motor 44, a rotation force transmission member 45, and a guiding tube holding portion 46.

The motor 44 generates driving force for rotating the helical tube 23 about the longitudinal axis thereof. The motor 44 is fixedly provided on, e.g., a side wall of the rotation portion main body 43. The motor 44 has a motor shaft 44a to which the rotation force transmission member 45 is integrally fixed.

The rotation force transmission member 45 is formed of an elastic resin member. The guiding tube holding portion 46 is placed opposed to the rotation force transmission member 45 fixed to the motor shaft 44a.

The guiding tube holding portion 46 is fixedly provided on, e.g., a bottom portion of the rotation portion main body 43. On a flat surface of the guiding tube holding portion 46 opposing to the rotation force transmission member 45 is formed a semicircular concave portion (not shown) that approximately agrees with the external shape of the helical tube 23 or the proximal-side component member 22.

The rotation mechanism portion 42 is configured such that the helical tube 23 constructing the introduction tube 20 is placed and held sandwiched between the rotation force transmission member 45 and the concave portion of the guiding tube holding portion 46.

Therefore, with the introduction tube 20, when the motor 44 is driven with the helical tube 23 placed between the rotation force transmission member 45 and the guiding tube holding portion 46, the rotation force transmission member 45 fixed to the motor shaft 44a is rotated, and the rotation driving force is transmitted to the helical tube 23 via the rotation force transmission member 45. The helical tube 23 thus transmitted with the rotation force rotates about the axis, with respect to the elastic cover tube 21 in the gap 23c formed between the inner circumferential surface of the helical shaped portion 23b and the elastic cover tube 21.

When the introduction tube 20 is inserted into a body cavity, the rotation of the helical tube 23 generates a propulsion force as that of a male screw moving with respect to a female screw, at a contacting portion between the helical shaped portion 23b and the intestine wall. This propulsion force causes the helical tube 23 to move toward the axial direction of the introduction tube 20 while rotating.

At this time, one end (the front end portion 23da) of the helical tube 23 is positionally restricted at a position to contact the convex portion 21d of the elastic cover tube 21, and the other end (the rear end portion 23db) at a position to contact the front surface portion 22e of the proximal-side component member 22. This results in the integration of the helical tube 23 and the elastic cover tube 21. Therefore, as the helical tube 23 moves, the elastic cover tube 21 moves in the same moving direction as that of the helical tube 23.

Also, at this time, in the introduction tube 20, the elastic cover tube 21 and the endoscope 2 are integrated by the fitting between the stopping convex portions 22b and the circumferential groove 12b, in a state shown in FIG. 3, that is, a state where the endoscope insertion portion 11 is inserted into the elastic cover tube 21 and the stopping convex portions 22b is fitted in the circumferential groove 12b.

Accordingly, the endoscope 2 moves in the same moving direction as that of the introduction tube 20 constructed by the helical tube 23 and the elastic cover tube 21, thereby moving toward the deep part of the intracavital canal.

As shown in FIG. 6, the introduction tube 20 has, around the outer circumferential surface of the insertion portion cover 10, the helical tube 23 having the helical shaped portion 23b formed in a clockwise winding toward the distal end side. The helical tube 23 forms the helical shaped portion 23b by winding the metal wire in a clockwise helical shape toward the distal end side. In other words, the helical tube 23 forms the helical shaped portion 23b by winding the metal wire in a helical shape in the same direction as that of a thread of a clockwise screw.

This results in that, in the introduction tube 20, the helical tube 23 is rotated clockwise about the longitudinal axis toward the insertion direction by the rotation device 40 to obtain propulsion force between the helical shaped portion 23b and the inner wall of the intracavital canal. Further, the helical tube 23 rotating clockwise about the longitudinal axis toward the insertion direction causes the Sigmoid colon portion to be drawn clockwise viewed from the front of the body and thus shortened, and the transverse colon upward in the canal and thus shortened, thereby generally linearizing the intestinal canal, which permits the introduction tube 20 to move forward to the deep part of the intracavital canal, as will be described later.

Now, operations of the endoscope system 1 constructed as described above will be described.

First, a medical staff (abbreviated as “staff”) prepares the endoscope 2 and the introduction tube 20 constructing the endoscope insertion-assisting apparatus 3. The staff moves the arm portion 41 of the rotation device 40 constructing the endoscope insertion-assisting apparatus 3 to place the rotation mechanism portion 42 at a desired position.

Next, the staff places a desired position of the helical tube 23 constructing the introduction tube 20 between the guiding tube holding portion 46 constructing the rotation mechanism portion 42 and the rotation force transmission member 45. This placement results in a state where the proximal end portion side of the introduction tube 20 is held by the rotation mechanism portion 42. At this time, the distal end portion side of the introduction tube 20 is placed, e.g., above a bed 7.

Then, the staff inserts and places the endoscope insertion portion 11 into the introduction tube 20 from the opening of the proximal-side component member 22 constructing the introduction tube 20. This results in a state where the endoscope 2 has the endoscope insertion portion 11 covered by the introduction tube 20, therewith completing the preparation for inserting the endoscope 2 into, e.g., the large intestine.

The staff also prepares the light source device 4, the video processor 5, and the monitor 6, which are peripheral devices, along with the preparation for the endoscope 2, the introduction tube 20, and the rotation device 40.

Next, steps for inserting the endoscope 2 covered by the introduction tube 20 into the large intestine will be described. First, as an insertion step, a surgeon (not shown) holds the distal end side of the introduction tube 20, and then inserts the distal end side of the introduction tube 20 into the large intestine from the anus of a patient 8 lying on the bed 7.

The introduction tube 20 with the distal end portion now inserted into the anus of the patient 8, has the helical shaped portion 23b formed on the external surface of the helical tube 23 in contact with the intestine wall. At this time, the relation between the helical shaped portion 23b and the intestine wall in contact to each other is that between male and female screws. Also, an endoscope image captured by the image-capturing element of the endoscope 2 is displayed on a screen of the monitor 6.

In the state where the helical shaped portion 23b is in contact with the intestine wall, the surgeon drives to rotate the motor 44 of the rotation mechanism portion 42 by a predetermined operation, as a rotation step. At this time, the surgeon operates to rotate and drive the motor 44 of the rotation mechanism portion 42 in a clockwise direction about the longitudinal axis in the insertion direction of the introduction tube 20.

In the rotation mechanism portion 42, driving the motor 44 to rotate clockwise causes the rotation force transmission member 45 to rotate clockwise via the motor shaft 44a. The rotation driving force of the rotation force transmission member 45 is transmitted to the helical tube 23 placed between the rotation force transmission member 45 and the guiding tube holding portion 46.

Thus, the helical tube 23 starts rotating clockwise about the longitudinal axis as shown in an arrow R in FIG. 7. At this time, at a contacting portion between the helical shaped portion 23b of the helical tube 23 which is rotating clockwise about the longitudinal axis and the intestine wall, there is a relation such as that of a clockwise screw moving with respect to a female screw, i.e., propulsion force for moving forward the helical tube 23 is generated.

As described above, the helical tube 23 has one end (the front end portion 23da) positionally restricted at a position to contact the convex portion 21d of the elastic cover tube 21, and the other end (the rear end portion 23db) at a position to contact the front surface portion 22e of the proximal-side component member 22, leading to integration between the helical tube 23 and the elastic cover tube 21. With this integration, the helical tube 23 is prevented from dropping off from the elastic cover tube 21, while contacting and pushing the rear surface portion 21dd of the convex portion 21d of the elastic cover tube 21 to move forward.

In this manner, the introduction tube 20 constructed by the helical tube 23 and the elastic cover tube 21 moves forward to the deep part in the large intestine by the propulsion force generated as a propulsion step.

At this time, the introduction tube 20 is integrated with the endoscope 2, because the proximal-side component member 22 of the introduction tube 20 has the stopping convex portions 22b fitted with the circumferential groove 12b. Therefore, as the introduction tube 20 moves, the endoscope 2 moves in the same direction thus being inserted into the deep part in the body cavity of the examinee.

In this state, when the surgeon makes an operation at hand such as to push forward the introduction tube 20, the introduction tube 20 with the endoscope insertion portion 11 inserted therein is introduced toward the intracavital deep part with a small amount of force. That is, the introduction tube 20 inserted from the anus 71 is moved from the rectum 72 toward the Sigmoid colon portion 73 by the propulsion force, at-hand operation and bending operation by the surgeon, or the like, with the endoscope insertion portion 11 being inserted in the introduction tube 20.

As shown in FIG. 8, when reaching the Sigmoid colon portion 73, the distal end of the introduction tube 20 contacts a bending portion by a scope, thus preventing the helical tube 23 from rotating.

As described above, the introduction tube 20 includes the helical tube 23 having the helical shaped portion 23b formed in a clockwise winding toward the distal end side, the helical tube 23 being provided around the outer circumference of the insertion portion cover 10.

For this reason, the rotated helical tube 23 is applied with a force as shown in an arrow shown in FIG. 9, thus twisting the introduction tube 20 in a clockwise direction as viewed from the front of the body.

As a result, as the linearization step, the introduction tube 20 draws the Sigmoid colon portion clockwise viewed from the front of the body to thereby shorten and generally linearize the same, while moving forward to the deep part of the intracavital canal, as shown in FIG. 10.

That is, in the present embodiment, an intracavital insertion method (large intestine insertion method) of the endoscope device includes the insertion step, the rotation step, the propulsion step, and the linearization step. This allows the introduction tube 20 to exhibit a sufficient propulsion function when being inserted into the large intestine, permitting for easy insertion of the endoscope insertion portion 11 into the deep part in the large intestine.

In some cases, the observation window member 24 of the introduction tube 20 is adhered with, e.g., a filth and the like. In this case, the surgeon press-operates the air and water supplying pressing button-switch 28 twice.

With the introduction tube 20, the air and water supplying device 27 is activated to supply water through the channel 21b to spout out, e.g., water from the opening of the air and water supplying nozzle 25 as indicated with the arrow shown in FIG. 3. In this manner, the introduction tube 20 can wash away the filth of the like adhering to the observation window member 24.

Also, the surgeon press-operates the air and water supplying pressing button-switch 28 once. In the introduction tube 20, the air and water supplying device 27 is activated to supply air through the channel 21b to spout out, e.g., air from the opening of the air and water supplying nozzle 25 as indicated with the arrow shown in FIG. 3. Thus, the introduction tube 20 can remove beads of moisture adhering on the surface of the observation window member 24. The surgeon also press-operates the suction pressing button-switch 29. The suction device is activated to suck body liquid and the like from the opening of the channel 21c, in the introduction tube 20.

Thereafter, the rotating introduction tube 20 passes through the Sigmoid colon portion 73 which is generally linearized as shown in FIG. 11, to further move forward to the descending colon portion 74 having low movability as shown in FIG. 12. The introduction tube 20 passes through the splenic flexure 76, which is an interface between the descending colon portion 74 and the transverse colon portion 75 having high movability, to smoothly move forward along the wall of the hepatic flexure 77 which is an interface between the transverse colon 75 and the ascending colon 78.

As shown in FIG. 13, when reaching the hepatic flexure 77, the distal end of the introduction tube 20 contacts a middle bending portion of the transverse colon, thus interrupting the rotation of the helical tube 23. At this time, the helical tube 23 being rotated is applied with forces shown in arrows so as to upwardly twist the introduction tube 20, as described referring to FIG. 9,

As a result, the introduction tube 20 generally draws upward as viewed from the front of the body and thus shortens the transverse colon 75, the splenic flexure 76, and the hepatic flexure 77, as shown in FIG. 14, as the linearization step.

After this step, though not shown, the introduction tube 20 moves forward, so that the distal end portion reaches, e.g., near the cecum portion 79 which is the destination position. The surgeon, on determining that the distal end portion of the introduction tube 20 has reached near the cecum portion 79 from an endoscope imaged displayed on the screen of the monitor 6, directs, e.g., a staff to stop the driving of the motor 44. The surgeon steps forward to pulling back the endoscope insertion portion 11 to perform large intestine endoscopy.

After the endoscopy is complete, the surgeon draws the endoscope insertion portion 11 out from the introduction tube 20 and discards the introduction tube 20. While at the same time, the surgeon inserts and places the endoscope insertion portion 11 into an unused new introduction tube 20. This permits the surgeon to perform the next inspection with the endoscope system 1 without cleaning and sterilizing the endoscope 2.

As described above, the introduction tube 20, which has around the outer circumference of the insertion portion cover 10 the helical tube 23 including the helical shaped portion 23b formed in a clockwise winding toward the distal end side, can move forward to the deep part in the body cavity, in that rotating the helical tube 23 clockwise about the longitudinal axis allows obtaining a propulsion force between the helical shaped portion 23b and the inner wall of the intracavital canal, while drawing the Sigmoid colon portion clockwise viewed from the front of the body to shorten the same, and drawing the transverse colon 75 upward the canal to shorten the same, thus generally linearizing the gut.

This makes it possible to insert the introduction tube 20 into the deep part in the large intestine along with the endoscope insertion portion 11, even if the introduction tube 20 does not have a very large total length, whereby providing a good operationality and reducing the production cost.

Also, the introduction tube 20 can surely prevent the endoscope insertion portion 11 from directly contacting the wall of the body cavity during inspection by inserting and placing the endoscope insertion portion 11 in the introduction tube 20. Accordingly, the staff is released from the trouble of cleaning and sterilizing the endoscope 2 and the introduction tube 20 every time an inspection is complete, by combining the endoscope 2 drawn out from the introduction tube 20 with a new introduction tube 20 for reuse after the inspection, instead of cleaning and sterilizing the endoscope 2.

Note that, although in the present embodiment, the rotation driving force of the motor 44 is transmitted to the proximal end side of the helical tube 23 which is a rotating cylindrical body in order to rotate the entire helical tube 23, the present invention is not limited thereto, but the rotation driving force of the motor 44 may be transmitted to, e.g., a middle portion or a distal end portion of the helical tube 23 to rotate the entire helical tube 23.

Further, although in the present embodiment, the insertion portion cover 10 that covers the endoscope insertion portion 11 is constructed as the insertion portion main body around which outer circumference being provided with the helical shaped portion 23b serving as the propulsion force generating portion, the present invention is not limited thereto, but the endoscope insertion portion may be the insertion portion main body, and a helical shaped portion be provided around the outer circumference of the endoscope insertion portion.

Furthermore, although in the present embodiment, the introduction tube is provided in the whole length with a helical tube that is wound clockwise, and the helical tube is rotated clockwise to perform both propulsion and linearization, the introduction tube may be constructed as shown in, e.g., FIGS. 15 and 16.

As shown in FIGS. 15 and 16, an introduction tube 20B, which is a modified example of the above-described embodiment, has a cylindrical helical portion 81 provided on a distal end of the elastic cover tube 21. The cylindrical helical portion 81 has an outer circumferential surface formed with a counterclockwise-wound helical shaped portion. The cylindrical helical portion 81 is rotatably constructed, connected to a rotation shaft (e.g., flexible shaft) 82 inserted into an insertion hole 83 of the elastic cover tube 21. The rotation shaft 82 is formed of multi-layers of metal wires knitted in a cylindrical mesh shape. The rotation shaft 82 has flexibility as well as rotation followability.

To a distal end portion 84 of the rotation shaft 82 is provided with a transmission gear 84a which engages with a transmission gear 81a formed on an inner circumferential surface of the cylindrical helical portion 81. A proximal end side of the rotation shaft 82 is rotatably constructed, connected to a rotation device not shown. Other constructions are almost the same as in the above-described embodiment.

The introduction tube 20B is constructed such that clockwise rotation of the rotation shaft 82 causes the cylindrical helical portion 81 having the counterclockwise-wound helical shaped portion to rotate clockwise.

With this construction, the introduction tube 20B can move forward to the deep part in the body cavity, in that rotating the helical tube 23 clockwise about the longitudinal axis allows obtaining a propulsion force between the helical shaped portion 23b and the inner wall of the intracavital canal, while drawing the Sigmoid colon portion clockwise viewed from the front of the body to shorten the same, and drawing the transverse colon upward the canal to shorten the same, thus generally linearizing the gut.

Note that, although in the modification example above, the cylindrical helical portion 81 and the rotation shaft 82 are constructed to rotate via a gear, the shaft and the propulsion portion may be integrated without the intermediary of the gear so that the rotation directions agree to each other.

The inventions described in the above embodiment are not limited to the embodiment and modification thereof, but can be embodied in other various modifications without departing from the spirit at the stage of practice. Further, the above-described embodiment includes various stages of inventions, and various inventions can be extracted from appropriate combinations of a plurality of disclosed components.

For example, if the problems mentioned in description of related art can be solved and the effects of the present invention recited in detailed description of preferred embodiments can be obtained even if a several components are deleted from all the components shown in the above-described embodiment, the construction deleted of the components can be extracted as an invention.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope device, comprising:
an insertion portion main body having flexibility which is insertable into an examinee's body;
a rotation propulsion portion rotatably placed around an outer circumference near a distal end of the insertion portion main body and centered around an axis of the insertion portion main body, the rotation propulsion portion having on an outer circumference thereof a helical shaped portion serving as a propulsion force generating portion; and
a rotation transmission shaft having flexibility for transmitting rotation force to the rotation propulsion portion from a proximal end side of the insertion portion main body, wherein
the helical shaped portion has a helix formed in a direction to exhibit a propulsion force to the direction of the distal end of the insertion portion when the rotation transmission shaft is rotated clockwise toward the distal end of the insertion portion main body.

2. The endoscope device according to claim 1, wherein the rotation transmission shaft is rotatably placed around the outer circumference of the insertion portion main body and is integrally formed with the rotation propulsion portion, the helical shaped portion being formed in a clockwise winding toward the direction of the distal end of the insertion portion.

3. The endoscope device according to claim 1, wherein the helical shaped portion of the rotation propulsion portion is provided over essentially the whole length of the rotation transmission shaft from the rotation propulsion portion.

4. The endoscope device according to claim 2, wherein the helical shaped portion of the rotation propulsion portion is provided over essentially the whole length of the rotation transmission shaft from the rotation propulsion portion.

5. An endoscope system, comprising:
an endoscope device including:
an insertion portion main body having flexibility which is insertable into an examinee's body;
a rotation propulsion portion rotatably placed around an outer circumference near a distal end of the insertion portion main body and centered around an axis of the insertion portion main body, the rotation propulsion portion having on an outer circumference thereof a helical shaped portion serving as a propulsion force generating portion; and
a rotation transmission shaft having flexibility for transmitting rotation force to the rotation propulsion portion from a proximal end side of the insertion portion main body; and
a rotation device for rotating the helical shaped portion of the endoscope device about a longitudinal axis of the helical shaped portion.

6. The endoscope system according to claim 5, wherein the rotation transmission shaft is rotatably placed around the outer circumference of the insertion portion main body and is integrally formed with the rotation propulsion portion, the helical shaped portion being formed in a clockwise winding toward the direction of the distal end of the insertion portion.

7. The endoscope system according to claim 5, wherein the helical shaped portion of the rotation propulsion portion is provided over essentially the whole length of the rotation transmission shaft from the rotation propulsion portion.

8. The endoscope system according to claim 6, wherein the helical shaped portion of the rotation propulsion portion is provided over essentially the whole length of the rotation transmission shaft from the rotation propulsion portion.

9. A method for inserting an endoscope device into a body cavity, the endoscope device including: an insertion portion main body having flexibility which is insertable into an examinee's body; a rotation propulsion portion rotatably placed around an outer circumference near a distal end of the insertion portion main body and centered around an axis of the insertion portion main body, the rotation propulsion portion having on an outer circumference thereof a helical shaped portion serving as a propulsion force generating portion; and a rotation transmission shaft having flexibility for transmitting rotation force to the rotation propulsion portion from a proximal end side of the insertion portion main body, wherein the helical shaped portion has a helix formed in a direction to exhibit a propulsion force to the direction of the distal end of the insertion portion when the rotation transmission shaft is rotated clockwise toward the distal end of the insertion portion main body, the method comprising:
inserting the endoscope device into an opening of an intracavital canal;
rotating the helical shaped portion of the endoscope device inserted into the opening of the intracavital canal, clockwise about a longitudinal axis of the insertion portion main body;
propelling the insertion portion main body toward a deep part of the intracavital canal, by obtaining a propulsion force between the helical shaped portion and an inner wall of the intracavital canal by the helical shaped portion which is rotating clockwise about the longitudinal axis of the insertion portion main body; and
generally linearizing the bending body cavity, by propelling the endoscope device toward the deep part of the intracavital canal while drawing the bending body cavity to right side of the examinee's body by means of friction action of the helical shaped portion rotating clockwise about the longitudinal direction of the insertion portion main body so as to shorten the bending body cavity.

10. The method for inserting an endoscope device into a body cavity according to claim 9, further comprising:
inserting the endoscope device from the anus to the rectum;
rotating the helical shaped portion of the endoscope device inserted into the rectum clockwise about a longitudinal axis of the insertion portion main body;
propelling the insertion portion main body toward a deep part of the large intestine, by obtaining a propulsion force between the helical shaped portion and an inner wall of the rectum by the helical shaped portion which is rotating clockwise about the longitudinal axis of the insertion portion main body; and
generally linearizing the Sigmoid colon, by propelling the endoscope device toward the deep part of the large intestine while drawing the Sigmoid colon to right side of the examinee's body by means of friction action of the helical shaped portion rotating clockwise about the longitudinal direction of the insertion portion main body so as to shorten the Sigmoid colon.

11. The method for inserting an endoscope device into a body cavity according to claim 10, further comprising:
generally linearizing the transverse colon, by propelling the endoscope device toward the deep part of the large intestine while drawing the transverse colon to upside of the examinee's body by means of friction action of the helical shaped portion rotating clockwise about the longitudinal direction of the insertion portion main body so as to shorten the transverse colon.
